(19) United States
(12) Patent Application Publication
(10) Pub. No.: US 2025/0195195 A1
(43) Pub. Date: Jun. 19, 2025

(54) TOOLS FOR FUSING TWO SECTIONS OF BONES AND PERFORMING OTHER MEDICAL PROCEDURES

(71) Applicant: THORAGENIX INNOVATIONS, INC., Boca Raton, FL (US)

(72) Inventors: Frederick J. THABET, Boca Raton, FL (US); Ravi KANAGALA, Boca Raton, FL (US); Keri GEORGE, Calgary (CA)

(73) Assignee: THORAGENIX INNOVATIONS, INC., Boca Raton, FL (US)

(21) Appl. No.: 18/849,880

(22) PCT Filed: Mar. 22, 2023
(86) PCT No.: PCT/US2023/015946

§ 371 Date: Sep. 23, 2024

Related U.S. Application Data
(60) Provisional application No. 63/323,367, filed on Mar. 24, 2022.

(51) Int. Cl. A61F 2/00; A61B 17/32; A61F 2/28

(52) U.S. Cl. CPC A61F 2/0095; A61B 17/32; A61B 2017/320064; A61F 2002/2835

(57) ABSTRACT

Systems, methods, and devices to fuse two sections of bone and perform other medical procedures are disclosed. A system includes a medical kit with tools and supplies, such as a sterile hydration pouch for hydrating an implant; a tissue extracting tool; and a topical biosensor for detecting an infection and/or temperature change. The tissue extracting tool has an extraction head with a suction pathway for pulling the tissue into a collection chamber. A depth gauge can protrude from a top surface or a bottom surface of the extraction head to define an insertion depth dimension of the extraction head which corresponds to a dimension of the implant (e.g., of a side protrusion of the implant). The tissue extracted with the tissue extracting tool can be used to hydrate the implant by injecting a saline solution including the tissue into a sterile hydration environment formed by the hydration pouch.

This application claims priority to U.S. Provisional Patent Application No. 63/323,367, filed Mar. 24, 2022, the entirety of which is herein incorporated by reference.

### BACKGROUND

### 1. Field

The present disclosure relates generally to systems, methods, and devices for performing medical procedures. In at least one example, the present disclosure relates to tools and supplies for fusing two sections of bone.

### 2. Description of Related Art

Physical intervention in tissue of a human or an animal, for example surgery, dates to prehistoric times. During a surgical procedure, an incision is made to access the interior of the body and when the procedure is complete, the incision is closed to the external environment. Some parts of the body are not directly accessible via a simple incision and present additional challenges. For example, organs such as the heart and lungs are protected within the ribcage/thoracic cage. Thus, to access a patient's heart, a surgeon often needs to separate the sternum. Upon completing the surgery, the incision is sutured closed.

The sternum or breastbone is a long, flat bone forming the middle portion of the front of the chest. Individual rib bones are connected along the sides of the sternum via cartilage to form the ribcage/thoracic cage which protects the heart, lungs, and major blood vessels from injury. The sternum is cut open in a sternotomy to gain access to the thoracic contents when performing cardiothoracic surgery.

A sternotomy is a surgical procedure in which a midline longitudinal incision is made through at least a portion of the sternum to allow opposing halves/portions to be laterally separated to provide access to organs within the ribcage/thoracic cage. When the surgical procedure is complete, the separated halves/portions are aligned with and secured to one another and the incision closed. A variety of devices, compositions, and methods for assisting with closing and healing of sternotomy wounds can be utilized.

The ideal goal for assisting with closing and healing of sternotomy wounds is complete rejoining of sternal portions with new bone growth in the absence of complications. Unfortunately, patient recovery from a conventional sternotomy is often slow and problematic. As the two sections (of the sternum) are brought back together by the surgeon, proper compression and a tight realignment of the end plates of the sternal surfaces is rarely achieved resulting in non-union, i.e. dehiscence, of separated sternal halves. This non-union allows for motion such as sliding of the surface of one sternal half against the surface of the other sternal half leading to significant pain for the patient and increased chance for development of infection. Additionally, the lack of proper compression leads to the formation of fibrous scar tissue instead of the desired new bone. If further surgical procedures are required, the scar tissue will have to be removed further complicating the procedure.

Serious complications can result from non-union of the two bones sections such as development of a deep sternal wound infection (DSWI), particularly within spaces left due to dehiscence of the two sections. DSWI after cardiac surgery and a high mortality rate and prolongs the hospital stay and significantly increases cost of care (e.g., up to $450,000). Various other surgeries face similar issues when attempting to rejoin the separated bone sections.

It is with these observations in mind, among others, that the presently disclosed technology was conceived.

### BRIEF SUMMARY

The presently disclosed technology addresses the foregoing problems by providing systems and methods for performing a medical procedure. For instance, the system can have a hydration pouch (e.g., container) with a hydration shell removably coupled to a planar hydration seal to enclose a volume inside the hydration pouch, forming a sterile hydration environment. An implant—or any other material to be hydrated, exposed to, contacted with, and/or partially or fully submerged in a solution—can be contained in the sterile hydration environment. The implant can have a first dimension, shape, profile, size, or other feature. Moreover, the hydration pouch can include a sealed port (e.g., a port with one or more seals separating the port from an external surface) disposed at a side of the hydration shell. The hydration shell can be a rigid hydration shell having a rectangular prism shape. Systems discussed herein can further includes a tissue extracting tool including an elongated gripping portion and an extraction head. The extraction head can be an extraction body or assembly at an end of the elongated gripping portion and/or can have a front portion with a second dimension (e.g., and/or size, shape, profile, or features) corresponding to the first dimension or feature of the implant. Additionally, a suction pathway can be formed between the front portion and a rear portion.

In some examples, the extraction head includes a tissue collection chamber in the suction pathway inside the extraction head. The extraction head can be a first type that includes a mounted blade couplable to the extraction head at a receiving area defined by a top surface and a back wall of the extraction head. The extraction head can be a second type that includes a front portion of the extraction head (e.g., an extractor head) that is rotatably couplable and decouplable from a body portion of the extraction head. The extraction head can include one or more protrusions from a top surface and/or a bottom surface, operable as a depth gauge. The depth gauge can define the second dimension of the front portion (e.g., corresponding to the first dimension of the implant) as a distance between a distal end of the front portion of the extraction head and a side of the depth gauge. Additionally or alternative, the extraction head can form an angle with the elongated gripping portion, the angle being between 30 degrees and 70 degrees. In some scenarios, the second dimension of the front portion causes the tissue extracting tool to form an indent in patient tissue having a shape corresponding to the implant (e.g., such that the implant fits snugly into the indent).

In some instances, the front portion of the extraction head includes a cut-out blade forming a collection opening behind the cut-out blade (e.g., as a tab of bent material cut from a base surface). Furthermore, a suction system can be couplable to the rear portion of the extraction head to provide the suction pathway, such as a path of air flow formed by the suction unit creating a vacuum through a hose connected to the rear portion of the extraction. The system can be a medical kit (e.g., with the different tools and supplies packaged and/or collected together for use together in an operating room). The medical kit can include a topical biosensor formed of a flexible strip with one or more sensor nodes connected to a digital display via one or more conductive paths embedded in the flexible strip.

In some examples, a medical kit for performing one or more medical procedures includes a hydration pouch with a rigid hydration shell removably coupled to a hydration seal forming a sterile hydration environment. The hydration pouch can also include an implant contained within the sterile hydration environment. The implant can have a first dimension, as discussed above. Moreover, a sealed port can be disposed at a side of the rigid hydration shell operable to be punctured with a needle, which can contain a saline solution and/or carrier component formed of tissue harvested from the patient receiving the medical operation. Furthermore, the medical kit can include a tissue extracting tool including an elongated gripping portion. The tissue extracting tool can also include an extraction head at an end of the elongated gripping portion, the extraction head having a front portion with a second dimension corresponding to the first dimension of the implant. Additionally, a suction pathway can be formed between the front portion and a rear portion.

In some instances, the sealed port includes a cap disposed over the sealed port and/or a silicon seal disposed between the cap and the sealed port. A removable cap seal can be disposed over an opening in the cap (e.g., a flexible material with adhesive such as a sticker). Additionally, the rigid hydration shell can be formed of a transparent or translucent material such that the implant is viewable during a hydration procedure. The medical kit can also include a flexible strip or a rigid staple, including an infection indicating portion and a temperature indicating portion, operable for placement at a suture location corresponding to the implant (e.g., after the implant is implanted and an incision at the suture location is sealed).

In some examples, a method for performing one or more medical procedure includes harvesting tissue from a patient using a tissue extracting tool. The tissue extracting tool can include the elongated gripping portion and the extraction head at the end of the tissue extracting tool. Furthermore a suction system can provide suction for pulling the tissue into the extraction head (e.g., through one or more collection openings formed behind cut-out blades). The method can additionally include hydrating or soaking an implant in a sterile hydration environment by injecting a saline solution, including the tissue harvested from the patient, into a hydration pouch containing the implant. In some instances, the method includes removing the implant from the hydration pouch by removing a hydration seal from a hydration shell forming the sterile hydration environment; and/or implanting the implant into the patient. Additionally or alternatively the patient can be monitored for an infection or a temperature change using a topical biosensor proximate to an incision location for the implant.

In some instances, the hydration seal has a rectangular prism shape and/or removing the hydration seal from the hydration shell includes peeling a planar side off of the rectangular prism shape. Additionally, harvesting the tissue can include cutting the tissue with a blade at a front of the extraction head and/or pulling the tissue, using the suction system, through an opening behind the blade into a collection chamber formed into the extraction head. Moreover, harvesting the tissue can involve extracting the tissue from the collection chamber. In some scenarios, extracting the tissue from the collection chamber includes unscrewing a front extracting portion of the extraction head, for instance, using threads on the front extracting portion that correspond to threads on a body portion of the extraction head. Additionally or alternatively, extracting the tissue from the collection chamber includes decoupling a bottom surface of the extraction head from a back wall of the extraction head.

The foregoing is intended to be illustrative and is not meant in a limiting sense. Many features of the embodiments may be employed with or without reference to other features of any of the embodiments. Additional aspects, advantages, and/or utilities of the presently disclosed technology will be set forth in part in the description that follows and, in part, will be apparent from the description, or may be learned by practice of the presently disclosed technology.

### BRIEF DESCRIPTION OF THE DRAWINGS

The foregoing summary, as well as the following detailed description, will be better understood when read in conjunction with the appended drawings. For the purpose of illustration, there is shown in the drawings certain embodiments of the disclosed subject matter. It should be understood, however, that the disclosed subject matter is not limited to the precise embodiments and features shown. The accompanying drawings, which are incorporated in and constitute a part of this specification, illustrate an implementation of systems and methods consistent with the disclosed subject matter and, together with the description, serves to explain advantages and principles consistent with the disclosed subject matter, in which:

FIG. 1 illustrates an example system including a medical kit with various tools and supplies for performing a medical procedure;

FIG. 2 illustrates an example system including a sterile hydration pouch, which can form at least a portion of the system depicted in FIG. 1;

FIG. 3 illustrates an exploded view of an example system including a sterile hydration pouch, which can form at least a portion of the system depicted in FIG. 1;

FIG. 4 illustrates an example system including a sterile hydration pouch using an implant hydration procedure, which can form at least a portion of the system depicted in FIG. 1;

FIG. 5 illustrates a section view of an example system including a sterile hydration pouch with an insertion port, which can form at least a portion of the system depicted in FIG. 1;

FIG. 6 illustrates an example system including a tissue extracting tool with a mounted blade, which can form at least a portion of the system depicted in FIG. 1;

FIG. 7 illustrates an exploded view of an example system including a tissue extracting tool, which can form at least a portion of the system depicted in FIG. 1;

FIG. 8 illustrates an example system including a tissue extracting tool with a curved paddle, which can form at least a portion of the system depicted in FIG. 1;

FIG. 9 illustrates an example system including a tissue extracting tool with an extraction head, which can form at least a portion of the system depicted in FIG. 1;

FIG. 10 illustrates an example system including a tissue extracting tool with an extraction head, which can form at least a portion of the system depicted in FIG. 1;

FIG. 11 illustrates an example topical biosensor device, which can form at least a portion of the system depicted in FIG. 1;

FIG. 12 illustrates an example topical biosensor strip with multiple sensing portions, which can form at least a portion of the system depicted in FIG. 1;

FIG. 13 illustrates an example topical biosensor staple with multiple sensing portions, which can form at least a portion of the system depicted in FIG. 1.

FIG. 14 illustrates an example method for performing one or more medical procedures using one or more tools, which can be performed by the system depicted in FIG. 1.

FIG. 15 illustrates an example method for performing one or more medical procedures using one or more tools including a hydration pouch, which can be performed the system depicted in FIG. 1;

FIG. 16 illustrates an example method for performing one or more medical procedures using one or more tools including an extracting tool, which can be performed by the system depicted in FIG. 1;

FIG. 17 illustrates an example environment for fusing two sections of bone using one or more tools, which can form at least a portion of the system depicted in FIG. 1; and

FIG. 18 illustrates an example environment for fusing two sections of bone using one or more tools, which can form at least a portion of the system depicted in FIG. 1.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the presently disclosed technology or the appended claims. Further, it should be understood that any one of the features of the presently disclosed technology may be used separately or in combination with other features. Other systems, methods, features, and advantages of the presently disclosed technology will be, or become, apparent to one with skill in the art upon examination of the figures and the detailed description. It is intended that all such additional systems, methods, features, and advantages be included within this description, be within the scope of the presently disclosed technology, and be protected by the accompanying claims.

The term “implant” or “surgical implant” can refer to a medical device manufactured to replace a missing biological structure, support a damaged biological structure, or enhance an existing biological structure.

The term “sternotomy” can refer to the surgical procedure of cutting a patient's sternum and separating cut portions to access thoracic organs such as the heart, lungs, and/or blood vessels. While the disclosed subject matter is focused on the sternum, it is foreseen that the system and method can be utilized in relation to any separated portions of any bone.

The term “patient” can include any human being or animal. The term “subject” may also be used herein to refer to the patient.

Cancellous bone can be a meshwork of spongy tissue of mature bone, for example found at the core of vertebral bones in the spine and/or at the ends of long bones such as the femur.

The term “bone growth-promoting material” can include any material that promotes and/or enhances bone growth both natural and synthetic. The term “bone growth-promoting agent” can include any composition or material that promotes and/or enhances bone growth. The bone growth-promoting agent added to the implant can be any known bone-growth promoting agent, including, but not limited to hydroxyapatite (HA), cellular growth factors, cytokines, silicates, and bone morphogenetic proteins (BMP). In some examples, strips of bone growth-promoting material, for example cancellous bone, fiber bone and/or collagen sponge, include one or more types of living cells. Living cells can also be carried by synthetic bone materials intended for the same purpose. The living cells added to the implant can be any living cells that promote and/or enhance bone growth including, but not limited to, stem cells, osteoblasts, osteoconductive cells, osteoinductive cells, and/or osteogenic cells. In some examples, strips of bone growth-promoting material, for example cancellous bone, fiber bone, synthetics, and/or collagen sponge, can include both bone growth-promoting agents and living cells as described herein.

The term “synthetic” material can include any man-made material. The synthetic material can be made from a combination of natural material and/or man-made or fabricated material.

Further, any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 1 mm includes all values from 0.1 mm to 9 mm. Additionally, the term “about” can refer to near or close to the desired dimension, for example “about” can refer to near or close to disclosed thicknesses and encompassed thicknesses that can be effectively implanted into the patient.

Further, as the presently disclosed technology is susceptible to embodiments of many different forms, it is intended that the present disclosure be considered as an example of the principles of the presently disclosed technology and not intended to limit the presently disclosed technology to the specific embodiments shown and described. Any one of the features of the presently disclosed technology may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, operation, action, or the like described in one embodiment may also be included in other embodiments, but is not necessarily included. Thus, the presently disclosed technology may include a variety of combinations and/or integrations of the embodiments described herein. Additionally, all aspects of the present disclosure, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps, operations, or acts are in some way inherently mutually exclusive.

### II. General Architecture

Systems disclosed herein improve upon previous techniques for fusing two section of bone and other medical procedures by using one or more tools, such as a sterile hydration pouch, a tissue extracting tool, and/or a topical biosensor. These tools and/or additional tools can form a medical kit or system used in an operating environment or operating room. The tissue extracting tool can be used for harvesting tissue from the patient which can be added to the implant using the sterile hydration pouch. Moreover, the tissue extracting tool can be used to create indents and/or reshape the interior tissue of the patient (e.g., cancellous bone tissue of the two bone section) to receive the implant. The shape of the implant can correspond to the shape of the indent created by the tissue extracting tool so that, when the implant is inserted into the indent, it fits well and snug. The system can also include a topical biosensor for placement on the patient after closing the incision/suture location for detecting an infection and/or temperature change occurring at the skin of the patient near the incision/suture location.

Performing one or more medical procedures using these tools results in multiple benefits, such as hemostasis, pain relief, realignment, setting the bone to fuse and heal, stabilizing the entire sternal construct. By harvesting the patient's own tissue for injection into the sterile hydration pouch, growth and healing is further promoted. Furthermore, by using the tissue extracting tool to shape the receiving area of the implant in the patient, gaps between the implant and the receiving the areas where bacteria can form and create infection are reduced or eliminated. Moreover, antibiotics, growth agents, pain medications, and other carrier components can be added to the saline solution injected into the sterile hydration pouch and absorbed by the implant, further improving patient outcomes. Additional benefits will become apparent from the disclosure herein.

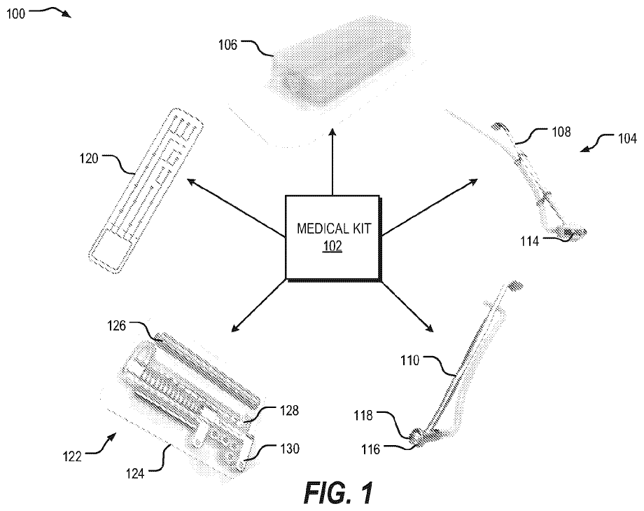

FIG. 1 illustrates an example system 100 including a medical kit 102 having one or more tool(s) and/or supplies 104 for performing one or more surgery operations. The one or more tool(s) and/or supplies 104 disclosed herein can be combined as the medical kit 102 (e.g., packaged, collected, and/or used together for a medical procedure) in any combination and/or can be used individually, resulting in multiple benefits, as discussed in greater detail below.

In some examples, the tool(s) and/or supplies 104 includes a sterile hydration pouch 106, a first tissue extracting tool 108, and/or a second tissue extracting tool 110. The first tissue extracting tool 108 can include a mounted blade 114 and the second tissue extracting tool 110 can include a mounted extraction head 116 with one or more blades 118. The tool(s) and/or supplies 104 can further include a topical biosensor 120 to indicate a temperature and/or an infection at the skin (e.g., at a suture site); and/or additional tools 122 such as a packaged bone fusing kit 124 including one or more of a sterile implant 126, a manipulator tool 128, and/or a spring-loaded clamp 130.

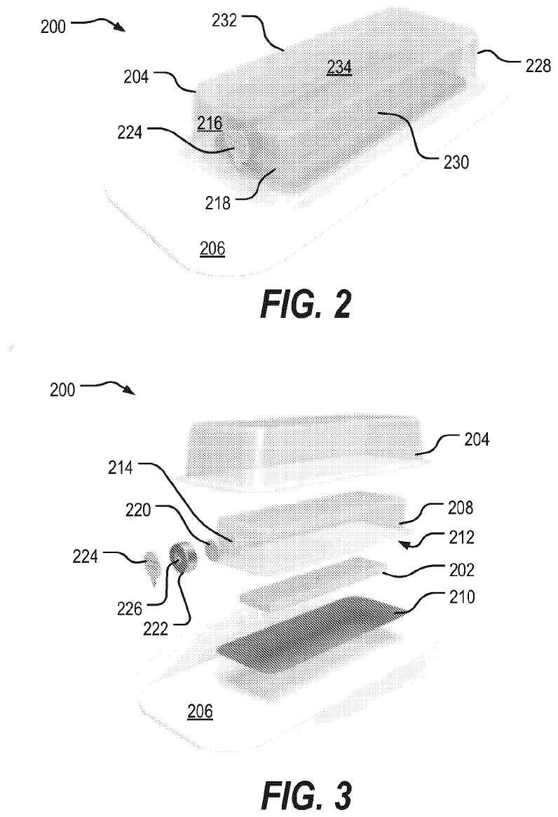

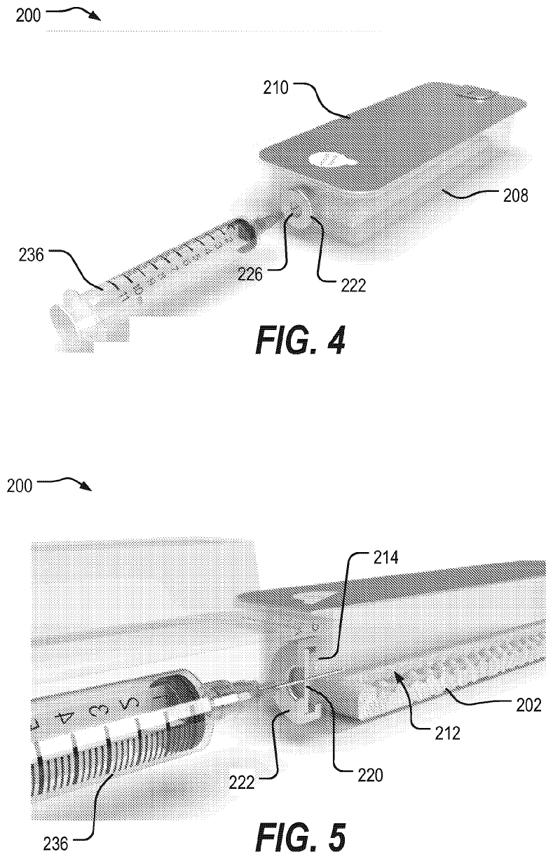

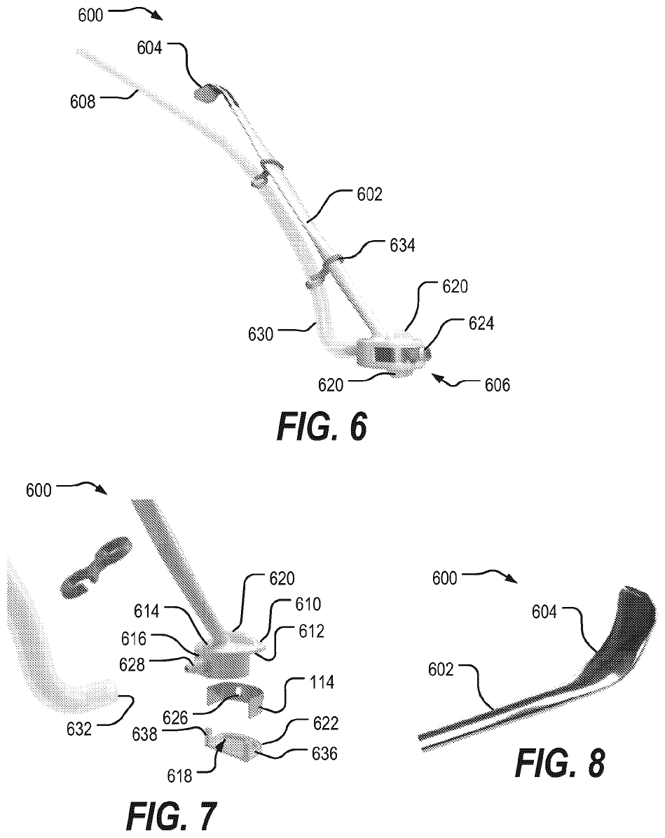

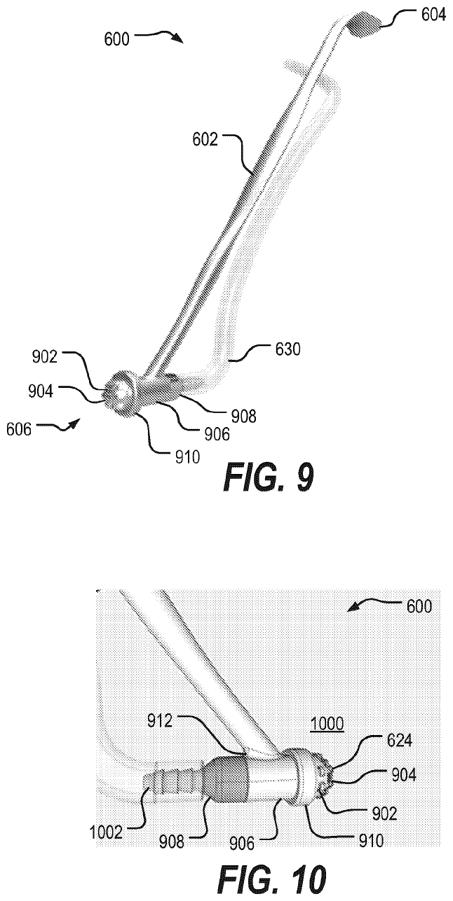

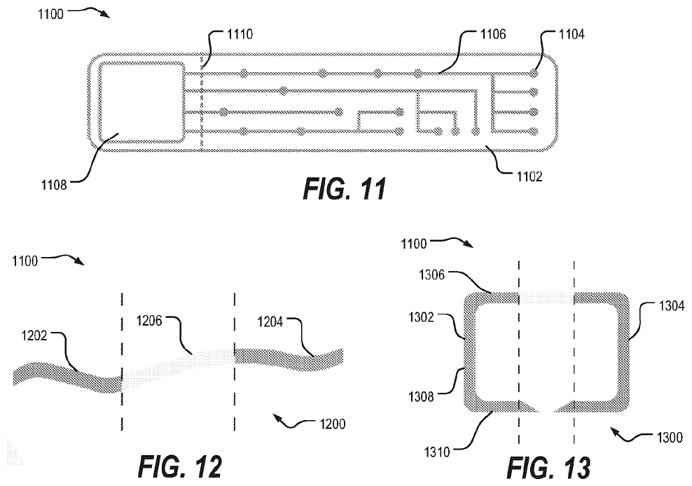

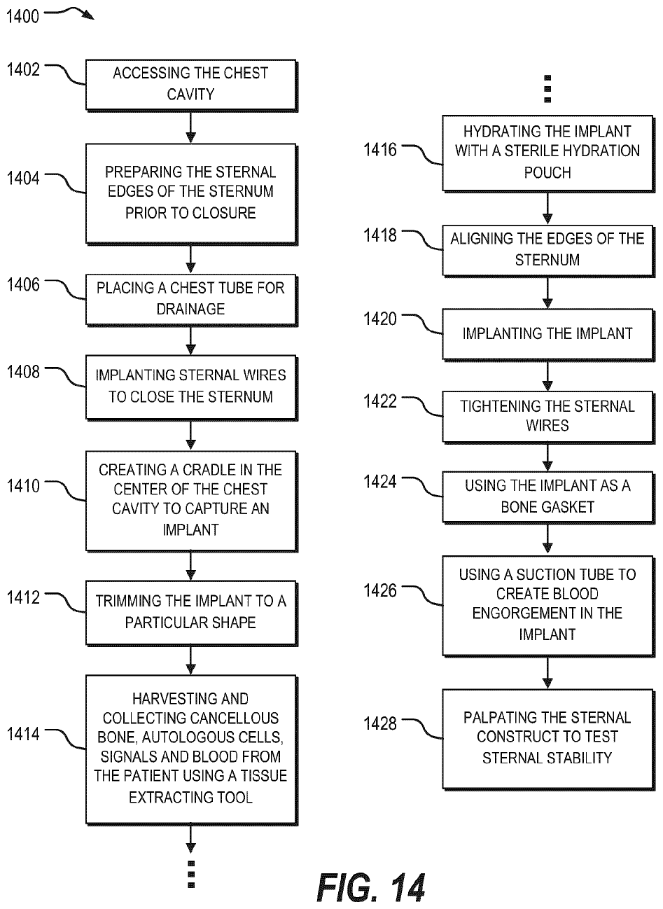

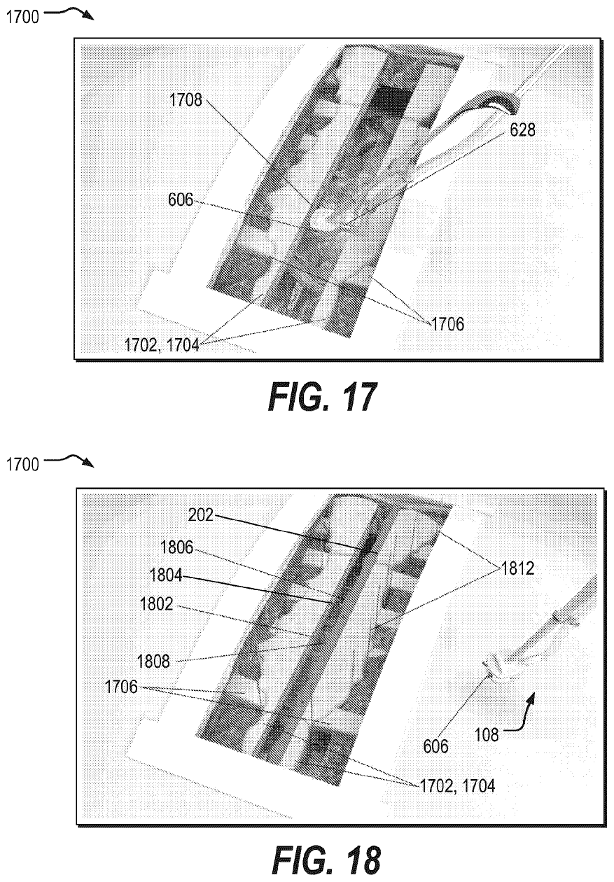

The tool(s) and/or supplies 104 are discussed in greater detail below. For instance, FIGS. 2-5 illustrates the sterile hydration pouch 106 and operations performed therewith. FIGS. 6-8 illustrate the first tissue extracting tool 108 and FIGS. 9 and 10 illustrate the second tissue extracting tool 110. FIGS. 11-13 illustrate the topical biosensor 112. FIGS. 14-16 illustrate methods for using one or more of the tool(s) and/or supplies 104. Finally, FIGS. 17 and 18 illustrate an example environment for using the tool(s) and/or supplies 104.

Although the tools and/or supplies 104 are discussed as forming a part of the medical kit 102, it is to be understood that the tools and/or supplies 104 can be used individually to perform improved medical operations. That is, the medical kit 102 can include only one of the tools and/or supplies 104 or a plurality of the tools and/or supplies 104. Furthermore, any combination of the tools and/or supplies 104 disclosed herein can be packaged (e.g., in one or more boxes, shrink wrap, blister packs, sterile pouches, etc.) and/or sold together or otherwise collected together (e.g., prior to performing the medical procedure) to form the medical kit 102.

Turning to FIGS. 2-5, a system 200 can include the sterile hydration pouch 106 for hydrating an implant 202, (e.g., or tissue or other synthetic material prior to insertion into a patient), which can form at least a portion of the system 100 depicted in FIG. 1.

In some instances, the sterile hydration pouch 106 is used to hydrate the implant 202 prior to inserting the implant between two sections of a severed bone. An outer packaging shell 204 can enclose and/or package the sterile hydration pouch 106 (e.g., for storage and/or shipping purposes. The outer packaging shell 204 can enclose a volume with a packaging back or planar surface 206 (e.g., formed of plastic, paper, cardboard, etc.) coupled to the outer packaging shell 204 to contain the sterile hydration pouch 106.

The sterile hydration pouch 106 can include a hydration shell 208 that couples to a hydration seal 210 to form an inner space 212 of the sterile hydration pouch 106. The hydration seal 210 can be removably coupled to the bottom edges of the hydration shell 208 using adhesive and/or a channel to receive an edge of the hydration seal 210. The hydration seal 210 can be a planar surface forming a bottom of the sterile hydration pouch 106 when the sterile hydration pouch 106 rests in a standing position (e.g., a position for use). Together the hydration shell 208 and the hydration seal 210 can form the inner space 212 that is fully isolated from an external environment of the sterile hydration pouch 106 with the hydration seal 210 defining the bottom of a volume of the inner space 212, and the hydration shell 208 defining a top and/or one or more sides of the volume of the inner space 212. This inner space 212 can form a sterile hydration environment for performing a hydration process with the implant inside the sterile hydration pouch 106.

The sterile hydration pouch 106 can include a port 214 formed into a first side 216 of the sterile hydration pouch 106 (e.g., at an end 218 of the sterile hydration pouch 106). The port 214 can include an opening closed with a port seal 220 (e.g., a silicone disk seal), a cap 222 over the port seal 220, and/or a removable cap seal 224. The removable cap seal 224 can be formed of a flexible material (e.g., paper or plastic) with an adhesive at one side for adhering over a cap opening 226.

The sterile hydration pouch 106 can include or be used with the implant 202, which can be formed of biological tissue material, synthetic material, or combinations thereof. For instance, the implant 202 can be the sterile implant 126 and/or can comprise any combination of cortical bone fibers, cancellous bone fibers, collagen sponge, cortical bone graft, synthetic bone, tissue graft, a porous material, a fibrous material, titanium, stainless steel, polyether ether ketone (PEEK), polyetherketone (PEK), surgical grade plastics of any kind, polyethylene, polymethylmethacrylate, vicryl surgical glues, synthetic surgical glues, porcelain, ceramics, and/or BioGlass. In some scenarios, the implant 202 can be packaged in the inner space 212 forming the sterile hydration environment of the implant 202. Additionally or alternatively, the implant 202 can be placed into the inner space 212 by medical personnel by removing and replacing the hydration seal 210.

In some instances, the implant 202 can have a shape or profile corresponding to a shape or profile of the sterile hydration pouch 106. For instance, the sterile hydration pouch 106 (e.g., the outer packaging shell 204 and/or the hydration shell 208) can have a rectangular prism shape with the first side 216 parallel to a second side 228 and perpendicular to a third side 230, a fourth side 232, and a top 234. As such, the implant 202 can also generally have a rectangular profile configured to fit within the rectangular prism formed by the sterile hydration pouch 106. Moreover, the implant 202 can have a round shape or curve corresponding to a shape of the sterile hydration pouch 106. Additionally or alternatively, the implant 202 can include one or more dimensions, features, shapes, profiles, protrusions, and/or combinations thereof that correspond to extraction head features of the first tissue extracting tool 108 and/or the second tissue extracting tool 110 (e.g., the mounted blade 114 and/or the extractor head 902). As such, the first tissue extracting tool 108 and/or the second tissue extracting tool 110 can be configured to create indents or imprints in tissue with dimensions or shapes that match the dimension or shape of the implant 202.

In some scenarios, the implant 202 can be hydrated or saturated prior to implantation using the sterile hydration pouch 106. For instance, a method to hydrate or saturate the implant 202 can include removing the removable cap seal 224 from the cap 222 to expose the cap opening 226. Then a needle 236 can puncture the hydration seal 210 through the cap opening 226. The needle 236 can contain a fluid such as a saline solution and/or one or more carrier components, which is injected into the inner space 212 forming the sterile hydration environment through the punctured hydration seal 210. After Injecting the saline solution and/or carrier components, the needle 236 can be withdrawn from the cap opening 226 and hydration seal 210 and the slightly compressed material of the hydration seal 210 around the needle 236 can naturally expand to fill the puncture opening left from the withdrawn needle 236, sealing it shut.

The sterile hydration pouch 106 can maintain a sterile and protected environment while the implant 202 hydrates. The implant 202 can be hydrated for a hydration period between 2 and 5 minutes or longer. During the hydration period, the implant 202 can absorb and/or become engorged with the saline solution and/or the carrier component(s). The carrier component(s) can include one or more of cancellous bone, cells, signals, and/or blood harvested from the patient. Additionally or alternatively, the carrier component(s) can include Plasma Rich Protein (PRP), antibiotics, Bone Morphogenic Protein (BMP), and other growth factors that are either naturally occurring, synthetic or harvested from donors or the patient, a cellular growth factor, a plasma rich protein, bone marrow aspirate, saline, bone morphogenetic proteins, mesenchymal stem cells, blood, osteoclasts, osteoblasts, antibiotics, analgesics, anticoagulants, beta-tricalcium phosphate, thrombin, surgical glues, demineralized bone matrix powders, collagen fibers, hemostatic medicine, and/or medications.

Once the implant 202 hydrates in the sterile hydration pouch 106 for the hydration period, the hydration seal 210—which provides a protective sterile covering for the inner space 212 during the hydration process—can be removed or peeled off the sterile hydration pouch 106. The surgeon or sterile staff member can retrieve the hydrated implant 202 from the inner space 212 and perform one or more medical operations with the implant 202, as discussed in greater detail below regarding FIGS. 14-18.

Turning to FIGS. 6-8, a system 600 can include the first tissue extracting tool 108 for extracting a tissue sample from a patient, which can form at least a portion of the system 100 depicted in FIG. 1.

In some examples, the first tissue extracting tool 108 can be used to collect a sample of tissue exposed during a medical operation. For instance, the first tissue extracting tool 108 can be used to collect soft tissue (e.g., muscle, skin, cancellous bone tissue, etc.), hard tissue (e.g., compact bone tissue), and/or fluids (e.g., blood, plasma, etc.). The first tissue extracting tool 108 can include an elongated gripping portion 602 with a curved paddle 604 at a first end and a tissue extractor portion 606 at a second end. The first tissue extracting tool 108 can also include a suction system 608 coupled to the tissue extractor portion 606.

In some examples, the tissue extractor portion 606 includes a head 610 with a receiving area 612 for receiving and/or coupling to the mounted blade 114. The receiving area 612 can be formed under a top surface 614 of the head 610 and can be at least partly defined by a back wall 616 of the head 610 extending down from the top surface 614. The mounted blade 114 can include a front face that curves or bends and, upon being mounted into the receiving area 612, at least partly defines (e.g., with the back wall 616 of the receiving area 612) a collection chamber 618 in the head 610. The tissue extractor portion 606 can further include a depth gauge 620, such as a protrusion extending from the top surface 614 and/or a bottom surface 622 for visibly measuring an insertion depth of the tissue extractor portion 606 and/or being a stop to abut a portion of bone at a predetermined depth to prevent further insertion.

The mounted blade 114 can include a cut-out blade 624 formed by a tab of the mounted blade material being cut and bent out from the mounted blade 114, and forming a collection opening 626 into the collection chamber 618. In some instances, the mounted blade 114 includes two cut-out blades 624 facing away from a front of the mounted blade 114, that is, curving forward. The mounted blade 114 can include three cut-out blades 624, four cut-out blades 624, or any number of cut-out blades 624. The openings in the planar material of the mounted blade 114 created behind the cut-out blades 624 can form the collection openings 626.

The first tissue extracting tool 108 can further include the suction system 608 which can be couplable to a suction hose connector 628 extending from the back wall 616 (e.g., in a backwards direction opposite from a direction towards the front surface of the mounted blade 114). The suction hose connector 628 can include an opening through a stem to communicatively couple the collection chamber 618 inside the tissue extractor portion 606 to a hose 630 of the suction system 608. For instance, an end 632 of the hose 630 can attach to the suction hose connector 628, for instance, by sliding over the suction hose connector 628 and being held in place with friction and/or one or more protrusions (e.g., ribs 1002 depicted in FIG. 10). The hose 630 can attach at another end to a surgical suction unit for providing suction (e.g., vacuum pressure) through the hose 630, and to the collection chamber 618 and the collection opening 626. Accordingly, a tissue sample abutting the front of the mounted blade 114 and/or being excised by the mounted blade 114 can be pulled through the collection opening 626 and into the collection chamber 618. A first size dimension (e.g., length, width, diameter, etc.) of the collection opening 626 can be greater than a second size dimension of an opening for the suction hose connector 628 formed into the back wall 616 such that the sample pulled through the collection opening 626 into the collection chamber 618 may be too large to pass through the suction hose connector 628, thus containing the tissue sample in the collection chamber 618. In other examples, a suction unit may be configured to pull the tissue sample through the suction hose connector 628 into another sample collection chamber fluidly coupled to the suction system 608. The first tissue extracting tool 108 can further include one or more holding clips 634 (e.g., two, three, four, etc.) for removably coupling the hose 630 to the elongated gripping portion 602. The one or more holding clips 634 can include a loop with a gap, such that the hose 630 can be pushed through the gap and, depending on the rigidity of the one or more holding clips 634, the gap may flex open and close (e.g., such that the loop acts as a living hinge). In this way, the hose 630 can be easily attached and detached from the first tissue extracting tool 108 with one hand (e.g., forming a one-handed configuration for attaching the suction system 608 to the first tissue extracting tool 108).

Additionally, the bottom surface 622 can form a bottom plane of the collection chamber 618 and can include a channel 636 for receiving a bottom edge of the mounted blade 114. The bottom surface 622 can include one or more vertical posts 638 for mating with the top surface 614 and side edges of the mounted blade 114 and/or the back wall 616. The bottom surface 622 can be removably coupled and easily attached and reattached to access the collection chamber 618 and remove the collected sample from the collection chamber 618.

In some instances, the first tissue extracting tool 108 includes the curved paddle 604 at the first end. The curved paddle 604 can bend away from an axis defined by the elongated gripping portion 602 and have a greater or flared width relative to a width of the elongated gripping portion 602. In other words, the curved paddle 604 can be a bent and flattened end of the elongated gripping portion 602. In some examples, the surgeon uses the curved paddle 604 to press into or scrape the tissue sample from the patient (e.g., cancellous tissue from an interior of a severed bone). For instance, the surgeon can use the curved paddle 604 and/or the tissue extractor portion 606 to form an indentation or impression into the cancellous tissue at least partially corresponding to a shape of the implant 202. Accordingly, the curved paddle 604 and/or tissue extractor portion 606 can be used to improve the ability of the implant 202 to fit into and mate with the cancellous tissue. When this occurs, the blood flow from the cancellous tissue into the porous/fibrous material of the implant 202 is increased, further improving integration of the implant 202 into the healing bone tissue and ultimately improving patient outcomes.

Turning to FIGS. 9 and 10, the system 600 can include the second tissue extracting tool 110 for extracting the tissue sample from the patient. Moreover, the second tissue extracting tool 110 can include any of the features discussed above regarding the first tissue extracting tool 108, and can form at least a portion of the system 100 depicted in FIG. 1.

The second tissue extracting tool 110 can include the elongated gripping portion 602 with the curved paddle 604 at the first end and the tissue extractor portion 606 at the second end. The tissue extractor portion 606 of the second tissue extracting tool 110 can have an extractor head 902 coupled to a front 904 of an extractor body 906. The suction hose connector 628 can protrude from a rear 908 of the extractor body 906.

The extractor head 902 can be a round or curved extractor head (e.g., having a hemisphere shape) and/or can have a pointed shape (e.g., like a pyramid). The extractor head 902 can include the cut-out blades 624 on an exterior surfacing facing forward. One or more collection opening 626 can be disposed near or behind the cut-out blades 624 to provide a pathway from an exterior 1000 of the extractor head 902 to an interior of the extractor head 902 (e.g., the collection chamber 618). The interior of the extractor head 902 can be communicatively coupled to an interior of the extractor body 906, which can further provide the pathway through the extractor body 906 to the rear 908 of the extractor body 906 and the suction hose connector 628. As such, the hose 630 of the suction system 608 can couple to the suction hose connector 628 (e.g., via a friction fit and/or one or more ribs 1002) to provide suction through the suction pathway such that tissue samples cut by the cut-out blades 624 are pulled through the collection opening 626 and into the extractor head 902.

In some examples, the extractor head 902 can be removably coupled to the front 904 of the extractor body 906. The extractor head 902 can be round with threaded screws for attaching to and detaching from the extractor body 906 (e.g., which can have a corresponding circular profile). As such, a sample of tissue pulled into the extractor body 906 by the suction system 608 can be retrieved by removing the extractor head 902 from the extractor body 906, pulling the sample of tissue from the interior of the extractor body 906 (e.g., the collection chamber 618), and attaching the extractor head 902 back onto the extractor body 906. The extractor head 902 can include a raised gripping portion 910 forming a lip around an end of the extractor head 902. The raised gripping portion 910 can also act as the depth gauge 620 and/or depth stop. Furthermore, the elongated gripping portion 602 can mate with the extractor body 906 at a juncture 912 forming an angle with a central axis of the extractor body 906. The angle can be an acute angle between 30 degrees and 70 degrees. The juncture 912 can be a fixed juncture such that the elongated gripping portion 602 and the extractor body 906 form an integrated unit, or the juncture 912 can be a removable or adjustable coupling so the elongated gripping portion 602 can be a detachable, movable, or separate component from the elongated gripping portion 602. In some instances, the juncture 912 can be an adjustable or pivotable juncture via a hinge, ball hinge, pivotable axis, or the like.

In some instances, the first tissue extracting tool 108 and/or the second tissue extracting tool 110 can be disassembled by removing the hose 630 from the suction hose connector 628 and/or opening the collection chamber 618 as discussed above. The first tissue extracting tool 108 and/or the second tissue extracting tool 110 can be assembled by securing the suction system 608 to the tool (e.g., using the holding clips 634 attached to the elongated gripping portion 602; and/or attaching the end 632 of the hose 630 to the suction hose connector 628).

The first tissue extracting tool 108 and/or the second tissue extracting tool 110 can be used to harvest and collect tissue from the patient and/or to form an indent, imprint, or impression into patient tissue by removing tissue from the patient. These functions simultaneously can be performed simultaneously. Moreover, the mounted blade 114 and/or the extractor head 902 can have a shape (e.g., a round shape, a depth dimension, a blade placement, etc.) and/or a stop (e.g., the depth gauge 620 and/or the raised gripping portion 910) that corresponds to a particular shape of an implant protrusion operable to mate with the indent (e.g., side protrusion 1808 of the implant 202, as depicted in FIG. 18). The first tissue extracting tool 108 and/or the second tissue extracting tool 110 can slice, shave, extract, remove, harvest, and/or collect cancellous bone, autologous cells, signals and blood from the patient.

Turning to FIGS. 11-13, an example system 1100 can include the topical biosensor 112 for visually indicating an epidermal status of the patient. FIG. 12 illustrates an example of a topical biosensor strip 1200 and FIG. 13 illustrates an example of a topical biosensor staple 1300, as discussed in greater detail below. The system 1100 can for at least a portion of the system 100 depicted in FIG. 1.

In some examples, the topical biosensor 112 can include a base or substrate formed of a flexible strip 1102 which can be a polymer-based and/or transparent material. The topical biosensor 112 can include one or more sensor node(s) 1104 embedded in the flexible strip 1102, and one or more conductive paths 1106 connecting the sensor node(s) 1104 to a digital display 1108. The topical biosensor 112 can include a folding line 1110 (e.g., a crease and/or a visual line indicator) traversing a width dimension of the topical biosensor 112. The digital display 1108 can be disposed on a first side of the topical biosensor 112 defined by the folding line 1110 and the sensor node(s) 1104 can be disposed on a second side defined by the folding line 1110 opposite the first side.

In some examples, the topical biosensor 112 can include multiple sections that perform different functions. For instance, the topical biosensor strip 1200 can include a first strip side section 1202 with an infection indicator, a second strip side section 1204 with a temperature indicator, and/or a middle strip section 1206 omitting any indicators (e.g., a control or neutral visualization). The infection indicator can include one or more substances reactive to common protein markers that are output by an epidermal infection, so when an infection occurs, the substances react to the protein markers of the infection and cause the first strip side section 1202 to change color. The temperature indicator can include one or more substances that react to a change in contact temperature by changing color. For instance, the temperature indicator can change color when a temperature changes a predetermined amount of degrees above body temperature (e.g., to 99°, 99.5°, 100°, 100.5°, 101°, or the like). The temperature indicator can change to multiple different colors to indicate multiple temperatures.

In some examples, the topical biosensor 112 can be the topical biosensor staple 1300 having a first staple side section 1302 with the infection indicator and/or a second staple side section 1304 with the temperature indicator. These staple side sections 1302 and/or 1304 can have a front side 1306 that can be exposed after implantation into the patient, and staple prongs 1308 that extend to and include a back side 1310 that can be submerged in the skin and/or underlying muscle tissue of the patient. As such an infection and/or a temperature change detection occurring at the back side 1310 can be transferred via the staple prongs 1308 up to the front side 1306, causing the visual indication to be exposed and visible to observers. In some instances, the topical biosensor 112 can be used as an operation for the medical procedure to fuse two sections of bone. For instance, upon suturing an incision in the skin, the topical biosensor 112 can be placed over or near the suture to provide infection and/or temperature (e.g., fever) monitoring at a site of the incision.

FIG. 14 illustrates an example method 1400 for fusing two sections of bone using the medical kit 102 and/or one or more of the tool(s) and/or supplies 104. The method 1400 can be performed by the system 100 depicted in FIG. 1.

At operation 1402, the method 1400 can include accessing the chest cavity. For instance a sternal saw may be used to form an incision in the sternum so that the breastbone can be retracted, exposing the heart, lungs, and vessels of the thoracic cavity. At operation 1404, the method 1400 can include preparing the sternal edges of the sternum prior to closure. For instance, once surgery on the soft tissues is completed, the surgeon can prepare the edges of the incised sternum to receive the implant 202. The edges of the incised sternum can vary from patient to patient depending on the age, overall health, and co-morbidities each patient is contending with. To bring a level of consistency into an environment that varies from patient to patient, a sternal edge preparation tool and/or the first tissue extracting tool 108 or the second tissue extracting tool 110 can be used to “adjust” and prepare the sternum to a point that is more consistent from patient to patient to receive the implant 202. At operation 1406, the method 1400 can include placing a chest tube for drainage. For instance, once the sternal edges have been prepared, the surgeon can place a chest tube into the chest cavity where it will remain temporarily to drain excess blood that collects in the chest cavity post operatively. At operation 1408, the method 1400 can include implanting sternal wires (e.g., sternal wires 1812 depicted in FIG. 18) to close the sternum. For instance, stainless steel sternal wires (e.g., or titanium) can be used to close the chest wall following open heart surgery. A series of 6 to 8 wires can be surgically implanted along the outer edges of the sternum from the manubrium, through the body of the sternum, to the xiphoid process, migrating around the edges of the sternum. At operation 1410, the method 1400 can include creating a cradle in the center of the chest cavity to capture the implant 202 (e.g., the sternal fusion graft). For instance, The “cradle” can be a pocket or space created by the effect the collective wires (e.g., the sternal wires 1812 in FIG. 18) as they are strategically implanted in the sternum. In other words, the cradle can be defined by a shape created by the sternal wires between the two sternal edges formed by the incision. The cradle can effectively capture implant 202 and keep the implant 202 from “falling” or slipping into the recesses of the chest cavity prior to final closure of the sternum. The cradle can also align the implant 202 and keep it centered against the edges of the sternum so that as the sternal edges are drawn together with the wire ties, the implant 202 maintains the proper orientation to the sternal edges so that optimum placement and seating of the implant 202 occurs.

Furthermore, at operation 1412, the method 1400 can include trimming the implant 202 (e.g., graft) to a particular shape. For instance, the implant 202 can be designed to be trimmed and adjusted by the surgeon to meet the unique and specific anatomic features of the individual patient's sternal anatomy. At operation 1414, the method 1400 can include harvesting and collecting cancellous bone, autologous cells, signals and blood from the patient. For instance, Cancellous bone, autologous blood, cells and signals can be harvested from the patient prior to implantation of implant 202, via sternal preparation using a collection tool (e.g., the first tissue extracting tool 108 and/or the second tissue extracting tool 110). The tool can hook up to the surgical suction system 608 and contain a bone trap and/or a reservoir (e.g., the collection chamber 618) to capture the shaved cancellous bone as well as the autologous cells, signals, and blood from the patient. At operation 1416, the method 1400 can include hydrating the implant 202 using the sterile hydration pouch 106. For instance, the implant 202 can be hydrated prior to implantation via the sterile hydration pouch designed to maintain a sterile and protected environment while the implant 202 hydrates. The sterile hydration pouch can include a port for receiving an injection of a saline solution and/or other solution. The time allotted to hydrate the graft may be no more than 2 to 5 minutes. The implant 202 can be hydrated in saline. The implant 202 can also be hydrated in a combination of the patient's own blood mixed with the “shaved” cancellous bone, cells and signals harvested from the patient during operation 1414. Additionally or alternatively, the implant 202 can be hydrated in Plasma Rich Protein (PRP), antibiotics, Bone Morphogenic Protein (BMP), and other growth factors that are either naturally occurring, synthetic or harvested from donors. Once the implant 202 has hydrated the predetermined amount of time, the implant 202 can be removed from the sterile hydration pouch 106 by peeling the protective sterile covering (e.g., the hydration seal 210) off the hydration shell 208 so the surgeon or sterile staff member can retrieve the implant 202. At operation 1418, the method 1400 can include aligning the edges of the sternum. Using the wire ties (e.g., the sternal wires 1812 in FIG. 18) as they are seated in the sternum, the surgeon can grasp all the ties on either side of the sternum and pull them all upward, away from the patient, moving the ties back and forth as the ties are pulled upward to further align the portions of the sternum prior to tightening the sternal wires down to close the sternal wound.

Additionally, at operation 1420, the method 1400 can include implanting the implant 202. For instance, once the sternal edges have been prepared and aligned, the surgeon can remove the implant 202 from the protective sterile pouch and place it on to the center of the sternal wire cradle. The leading edge of the graft can rest directly on the wires of the cradle. The implant 202 can rest in this position until the surgeon does the final tightening of the wire ties to close the sternal bone. At operation 1422, the method 1400 can include tightening the sternal wires. For instance, when the surgeon is ready to tighten the sternal wire construct, the surgeon can begin with one set of wire ties at a time. This operation can start at the set of ties at the top of the manubrium, and include grasping the ties in both hands and twisting the ties together in either a clockwise or counterclockwise direction, dependent on the choice of the surgeon. This action can mimic the same motion that someone may use while twisting the tie to a loaf of bread. Additionally or alternatively, the wires can include a closing mechanism attached at their ends (e.g., a clamp). The surgeon can start at the top of the manubrium and work his/her way down the sternum until all the wire ties have been twisted shut in this manner. Then the surgeon can take a needle driver, grasp the end of the twisted wire construct, and do a final tightening of the wires. Once the surgeon has performed the final tightening on all the individual wire constructs, he/she can take a pair of wire cutters and clip the excess wire from the wire construct. The surgeon can then turn the end of the wire construct down and dip the sharp tip of the wire construct into the bone of the patient so that the tip will not protrude through the skin post surgically. According to the surgeon's preference, each wire construct can be either a single or a double set of wires. Moreover, techniques disclosed herein can use the wires to create a lattice (e.g., similar to rebar in cement) to add structure to the internal bracing system or sternal structure formed by the wires, with the wires anchored into the internal bracing system. In some instances, the implant 202 (e.g., the graft) and the closing mechanisms (e.g., the wires can be pre-attached to form a single unit prior to implantation. Tightening and securing the wire ties can create a closed sternal construct including the wire ties, the implant 202, and the two sections of bone.

Furthermore, at operation 1424, the method 1400 can include using the implant 202 as a bone gasket. For instance, the implant 202 can act like a “bone gasket” to help seal and align the sternum. This strong seal can reduce blood loss, mitigate pain, and increase the overall integrity of the sternal construct while eliminating micromovement and translation, and preparing the edges of the sternal bone to fuse back together. At operation 1426, the method 1400 can include using the suction tube to create blood engorgement into the implant 202. For instance, after the implant 202 has been implanted and before the surgeon closes the skin incision, the surgeon can take a suction tip and holds it onto the implanted implant 202 and draw blood into the implant 202 from the edge of the sternum. If the implant 202 was hydrated in saline, this can give the surgeon the ability to draw blood into the implant 202 that is rich with the cells and signals from the sternum that encourage bone growth. At operation 1428, the method 1400 can include palpating the sternal construct to test sternal stability. For instance, as a postoperative test to validate the stability of the sternal construct, a surgeon can take the palm of their hand and press down gently but firmly into the sternum. The motion of applying downward pressure then releasing that pressure can cause the sternum to move (e.g., “book”) slightly open and closed. Without the implant 202 in place, this test would cause the patient enormous amounts of pain and on a scale between 1 to 10 it would cause the patient's pain to jump to a 10 out of 10 level. With the implant 202 in place the security and integrity of the sternal construct can go up exponentially. The patient's pain can go from a 9 or 10 on the pain scale down to a 0 or 1.

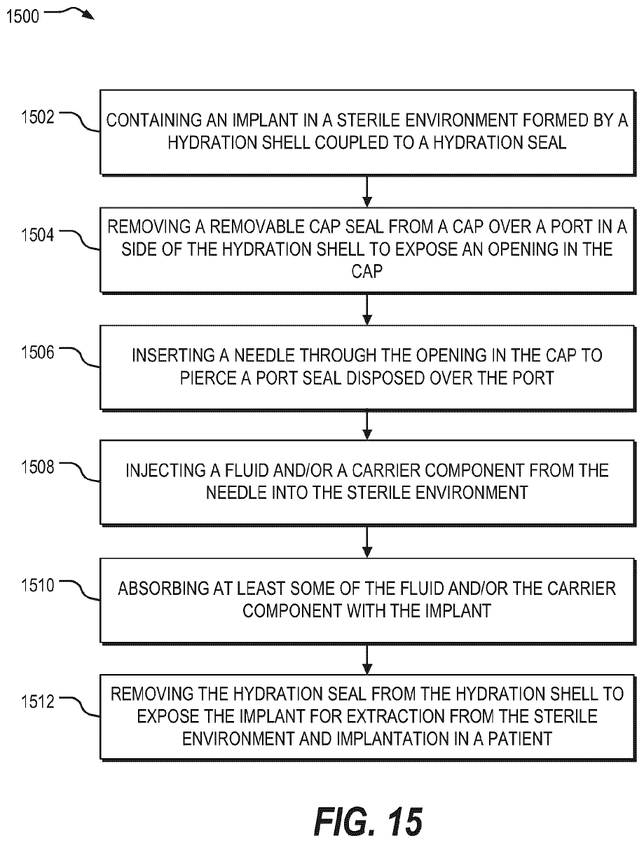

FIG. 15 illustrates an example method 1500 for using the sterile hydration pouch 106 to perform a medical procedure. The method 1500 can form a portion of the method 1400 (e.g., at operation 1416) and/or can be performed by the system 100 depicted in FIG. 1.

At operation 1502, the method 1500 contains an implant in a sterile environment formed by a hydration shell coupled to a hydration seal. At operation 1504, the method 1500 removes a removable cap seal from a cap over a port in a side of the hydration shell to expose an opening in the cap. At operation 1506, the method 1500 inserts a needle through the opening in the cap to pierce a port seal disposed over the port. At operation 1508, the method 1500 injects a fluid and/or a carrier component from the needle into the sterile environment. At operation 1510, the method 1500 absorbs at least some of the fluid and/or the carrier component with the implant. At operation 1512, the method 1500 removes the hydration seal from the hydration shell to expose the implant for extraction from the sterile environment and implantation in a patient.

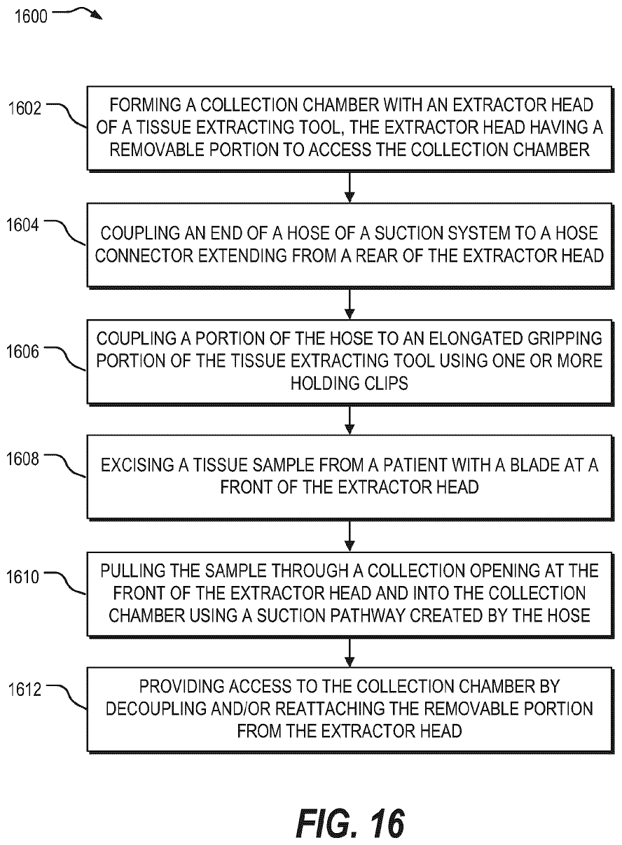

FIG. 16 illustrates an example method 1600 for using a tissue extracting tool (e.g., the first tissue extracting tool 108 and/or the second tissue extracting tool 110) to perform a medical procedure, such as a sample extraction procedure. The method 1600 can form at least a portion of the method 1400 (e.g., at operation 1414) and/or can be performed by the system 100 depicted in FIG. 1.

At operation 1602, the method 1600 forms a collection chamber with an extractor head of a tissue extracting tool, the extractor head having a removable portion to access the collection chamber. At operation 1604, the method 1600 couples an end of a hose of a suction system to a hose connector extending from a rear of the extractor head. At operation 1606, the method 1600 couples a portion of the hose to an elongated gripping portion of the tissue extracting tool using one or more holding clips. At operation 1608, the method 1600 excises a tissue sample from a patient with a blade at a front of the extractor head. At operation 1610, the method 1600 pulls the sample through a collection opening at the front of the extractor head and into the collection chamber using a suction pathway created by the hose. At operation 1612, the method 1600 provides access to the collection chamber by decoupling and/or reattaching the removable portion from the extractor head.

It is to be understood that the specific order or hierarchy of operations in the method depicted in FIGS. 14-16 and throughout this disclosure are instances of example approaches and can be rearranged while remaining within the disclosed subject matter. For instance, any of the operations depicted in FIGS. 14-16 and throughout this disclosure can be omitted, repeated, performed in parallel, performed in a different order, and/or combined with any other of the operations depicted in FIGS. 14-16 and throughout this disclosure. Moreover, any of the example systems, methods or environments illustrated in FIGS. 1-18, or the components or operations thereof, can be combined together.

FIGS. 17 and 18 illustrate an example environment 1700 for using the tools and/or supplies 104. For instance, the environment 1700 can be an operating room environment for the various medical procedures discussed herein.

In some instances, the environment 1700 includes a first section of bone 1702 (e.g., a first sternum half) separate from a second section of bone 1704. Furthermore, one or more ribs 1706 can attach to the bone 1702 and/or the bone 1704. With the bones 1702 and 1704 separated and exposing inner bone tissue (e.g., cancellous bone tissue), operation 1414 of the method 1400 can be performed with the first tissue extracting tool 108 and/or the second tissue extracting tool 110. For instance, a method performed at the 1700 can include pushing the mounted blade 114 and/or the extractor head 902 into the cancellous tissue 1708 until an edge of the bone 1702 abuts the depth gauge 620 and/or the raised gripping portion 910, causing the extraction process to stop at a predetermined depth (e.g., a distance between the depth gauge 620/raised gripping portion 910 and the cut-out blades 624).

In some examples, as shown in FIG. 18, the first tissue extracting tool 108 and/or tool 110 can form one or more indentation(s) 1802 with a shape 1804 that correspond to a shape of the front of the tissue extractor portion 606. Furthermore this shape 1804 can correspond to a second shape 1806 of a protrusion 1808 of the implant 202 (e.g., extending from a side 1810 of the implant 202).

In some examples, upon forming the indentation 1802 in the cancellous tissue (e.g., or other patient tissue), one or more sternal wire(s) 1812 wrapped around the first section of bone 1702, the implant 202, and the second section of bone 1704 can be pulled tight and twisted together across a front of the sternum to compress the implant 202 between the first section of bone 1702 and the second section of bone 1704 to form the sternal construct, as discussed above.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A system for performing a medical procedure, the system comprising:
a hydration pouch including:
a hydration shell removably coupled to a planar hydration seal to enclose a volume forming a sterile hydration environment;
an implant contained in the sterile hydration environment, the implant having a first dimension; and
a sealed port disposed at a side of the hydration shell; and
a tissue extracting tool including:
an elongated gripping portion; and
an extraction head at an end of the elongated gripping portion, the extraction head having a front portion with a second dimension corresponding to the first dimension of the implant, and a suction pathway between the front portion and a rear portion.

2. The system of claim 1,
wherein,
the extraction head includes a tissue collection chamber in the suction pathway.

3. The system of claim 1,
wherein,
the extraction head includes a mounted blade couplable to the extraction head at a receiving area defined by a top surface and a back wall of the extraction head.

4. The system of claim 1,
wherein,
the extraction head includes a protrusion from a top surface, operable as a depth gauge, defining the second dimension of the front portion corresponding to the first dimension of the implant.

5. The system of claim 1,
wherein,
the extraction head forms an angle with the elongated gripping portion, the angle being between 30 degrees and 70 degrees.

6. The system of claim 1,
wherein,
the second dimension of the front portion causes the tissue extracting tool to form an indent, in patient tissue, having a shape corresponding to the implant.

7. The system of claim 1,
wherein,
the front portion of the extraction head includes a cut-out blade forming a collection opening behind the cut-out blade.

8. The system of claim 1,
further comprising,
a suction system couplable to the rear portion of the extraction head to provide the suction pathway.

9. The system of claim 1,
wherein,
the system is a medical kit that further includes a topical biosensor formed of a flexible strip with one or more sensor nodes connected to a digital display via one or more conductive paths embedded in the flexible strip.

10. The system of claim 1,
wherein,
the hydration shell is a rigid hydration shell having a rectangular prism shape.

11. A medical kit device for performing a medical procedure, the medical kit device comprising:
a hydration pouch including:
a rigid hydration shell removably coupled to a hydration seal forming a sterile hydration environment;
an implant contained within the sterile hydration environment, the implant having a first dimension; and
a sealed port disposed at a side of the rigid hydration shell operable to be punctured with a needle; and
a tissue extracting tool including:
an elongated gripping portion; and
an extraction head at an end of the elongated gripping portion, the extraction head having a front portion with a second dimension corresponding to the first dimension of the implant; and a suction pathway between the front portion and a rear portion.

12. The medical kit device of claim 11,
wherein,
the sealed port includes a cap disposed over the sealed port with a silicon seal disposed between the cap and the sealed port.

13. The medical kit device of claim 12,
further comprising,
a removable cap seal disposed over an opening in the cap.

14. The medical kit device of claim 11,
wherein,
the rigid hydration shell is formed of a transparent or translucent material.

15. The medical kit device of claim 11,
further comprising,
a flexible strip or a rigid staple, including an infection indicating portion and a temperature indicating portion, operable for placement at a suture location corresponding to the implant.

16. A method for performing a medical procedure, the method comprising:
harvesting tissue from a patient using a tissue extracting tool including an elongated gripping portion and an extraction head at an end of the tissue extracting tool, a suction system providing suction for pulling the tissue into the extraction head;
hydrating an implant in a sterile hydration environment by injecting a saline solution, including the tissue harvested from the patient, into a hydration pouch containing the implant;
removing the implant from the hydration pouch by removing a hydration seal from a hydration shell forming the sterile hydration environment;
implanting the implant into the patient; and
monitoring the patient for an infection or a temperature change using a topical biosensor proximate to an incision location for the implant.

17. The method of claim 16,
wherein,
the hydration seal has a rectangular prism shape; and
removing the hydration seal from the hydration shell includes peeling a planar side off of the rectangular prism shape.

18. The method of claim 17,
wherein,
harvesting the tissue includes:
cutting the tissue with a blade at a front of the extraction head;
pulling the tissue, using the suction system, through an opening behind the blade into a collection chamber formed into the extraction head; and
extracting the tissue from the collection chamber.

19. The method of claim 18,
wherein,
extracting the tissue from the collection chamber includes unscrewing a front extracting portion of the extraction head.

20. The method of claim 18,
wherein,
extracting the tissue from the collection chamber includes decoupling a bottom surface of the extraction head from a back wall of the extraction head.
